(19) United States
(12) Patent Application Publication
(10) Pub. No.: US 2010/0267039 A1
(43) Pub. Date: Oct. 21, 2010

(54) METHOD OF DIFFERENTIATING COCCIDIOIDES SPECIES

(75) Inventors: Elizabeth Driebe, Flagstaff, AZ (US); Paul Keim, Flagstaff, AZ (US); Kelly Sheff, Flagstaff, AZ (US); David Engelthaler, Flagstaff, AZ (US)

Correspondence Address: The Translational Genomics Research Institute, 445 N. 5th Street, Phoenix, AZ, 85004, US

(73) Assignees: Translational Genomics Research Institute (TGen), Phoenix, AZ (US); Arizona Board of Regents - Northern Arizona University, Flagstaff, AZ (US)

(21) Appl. No.: 12/764,833

(22) Filed: Apr. 21, 2010

Related U.S. Application Data
(60) Provisional application No. 61/171,367, filed on Apr. 21, 2009.

(51) Int. Cl. C12Q 1/68

(52) U.S. Cl. 435/6

(57) ABSTRACT

The invention pertains to methods and kits useful in the identification of and differentiation between Coccidioides immitis and Coccidioides posadasii. The methods involve the addition of oligonucleotides to mixtures containing nucleic acid isolated from a sample and performing nucleic acid amplification on the mixture.

### BACKGROUND OF THE INVENTION

Generally, the invention falls under the field of differentially identifying pathogen species, specifically species of Coccidioides organisms that cause Valley Fever. Coccidioidomycosis is caused by infection with Coccidioides immitis or C. posadasii. Coccidioides immitis and C. posadasii are the fungal etiologic agents of coccidioidomycosis (aka Valley Fever) and are endemic to arid soils of the southwest United States, as well as parts of Mexico, and Central and South America (See Reference 1). Primary hosts acquire Coccidioides via inhalation of aerosolized arthroconidia upon soil disruption (See Reference 2). Coccidioidomycosis most commonly causes a progressive pulmonary infection in humans and other vertebrate hosts but also can disseminate to other body parts including the skin, brain, bone, and meninges. This disseminated secondary coccidioidomycosis often is severe and can result in patient death (See Reference 3). However, in cases where infection is resolved patients usually acquire a specific and lifelong immunity to the fungus (See Reference 4).

Coccidioidomycosis infection rates have increased dramatically in the last decade with the state of Arizona reporting the number of reported cases per 100,000 population having increased from 20.8 in 1997 to 86.1 in 2006 (See Reference 5). Potential causes for this increase include influxes of immunologically naïve individuals into Arizona (See References 6 and 7). A significant number of individuals from outside the Coccidioides endemic region migrate annually to the desert Southwest and are at greater risk for development of coccidioidomycosis, even after return to their respective homes. These infections, therefore, are likely to escape or confound diagnosis in non-endemic regions (See Reference 7).

While RTPCR based assays have been developed that help clinicians identify Coccidioides as a cause of illness (See Reference 8), such assays are unable to differentiate one Coccidioides species from another. Such information is vital for epidemiological and research purposes in that it allows identification of the location of infection and ask the question of whether differential clinical outcomes result from infection with one Coccidioides species versus another. Population influx in Coccidioides-endemic areas may contribute to rate of infection increases not only because there are additional individuals relocating to these areas but also because there is increased new home construction in virgin desert areas, and subsequent soil disturbances.

While it is thought that both C. immitis and C. posadasii infections cause the same clinical symptoms in the host (See Reference 2), little, if any research has been conducted comparing specific disease outcomes with a particular Coccidioides species. Further, capacity for genotypic characterization of Coccidioides species for epidemiological tracking of disease transmission has been limited (See Reference 4). Generally, it is recognized that C. immitis appears primarily confined to the San Joaquin Valley and surrounding areas in California, while C. posadasii is found in other Southwestern states, Central Mexico, and certain arid regions of South America (See Reference 4). Until recently, only microsatellite analysis has been used to distinguish the two species of Coccidioides (See References 4 and 9), however, this method is both costly and time consuming. Clearly, new methods of differentiating between Coccidioides species are necessary.

### BRIEF SUMMARY OF THE INVENTION

The present invention provides among other things a method of identifying and differentiating between different Coccidioides species.

It is an object of the invention to provide an assay that can identify the source of an unknown infection as either C. immitis or C. posadasii.

It is an object of the invention to facilitate research into differences in pathology, outcome and treatment between Coccidioides species.

It is an object of the invention to provide an assay that rapidly differentiates C. immitis from C. posadasii.

It is an object of the invention to provide an assay that inexpensively differentiates C. immitis from C. posadasii.

It is an object of the invention to determine the presence of C. immitis in a sample.

It is an object of the invention to determine the presence of C. posadasii in a sample.

It is an object of the invention to determine whether or not a sample contains either C. immitis or C. posadasii.

It is an object of the invention to provide a kit that facilitates determining whether or not a sample contains either C. immitis or C. posadasii.

The above and other objects may be achieved through the use of methods involving receiving a sample, isolating nucleic acid from the sample, adding a first oligonucleotide including a sequence selected from the group consisting of SEQ ID NO. 1, SEQ ID NO. 2, SEQ ID NO. 3, SEQ ID NO. 4, SEQ ID NO. 9, SEQ ID NO. 10, SEQ ID NO. 11, SEQ ID NO. 12, SEQ ID NO. 13, SEQ ID NO. 14, SEQ ID NO. 15, and SEQ ID NO. 16 to a mixture comprising the nucleic acid; subjecting the mixture to conditions that allow nucleic acid amplification and identifying the sample as containing C. immitis or not containing C. immitis on the basis of a result of the nucleic acid amplification. If the first nucleic acid includes SEQ ID NO. 9 then the method may further comprise adding a second oligonucleotide including SEQ ID NO. 10 to the mixture. This aspect of the method may further comprise performing DNA sequencing on a product of the nucleic acid amplification or it may further comprise adding a third oligonucleotide including SEQ ID NO. 1 to the mixture. If the first nucleic acid includes SEQ ID NO. 11 then the method may further comprise adding a second oligonucleotide including SEQ ID NO. 12 to the mixture. This aspect of the method may further comprise performing DNA sequencing on a product of the nucleic acid amplification or it may further comprise adding a third oligonucleotide including SEQ ID NO. 2 to the mixture. If the first nucleic acid includes SEQ ID NO. 13 then the method may further comprise adding a second oligonucleotide including SEQ ID NO. 14 to the mixture. This aspect of the method may further comprise performing DNA sequencing on a product of the nucleic acid amplification or it may further comprise adding a third oligonucleotide including SEQ ID NO. 3 to the mixture. If the first nucleic acid includes SEQ ID NO. 15 then the method may further comprise adding a second oligonucleotide including SEQ ID NO. 16 to the mixture. This aspect of the method may further comprise performing DNA sequencing on a product of the nucleic acid amplification or it may further comprise adding a third oligonucleotide including SEQ ID NO. 4 to the mixture. In any of the above aspects, the third oligonucleotide may comprise a label. The label may be any label including a fluorescent label such as FAM, dR110, 5-FAM, 6FAM, dR6G, JOE, HEX, VIC, TET, dTAMRA, TAMRA, NED, dROX, PET, BHQ+, Gold540, or LIZ. The first oligonucleotide may be affixed to a solid substrate. The sample may comprise an environmental sample. In other aspects, the sample may be derived from a subject. A subject may be a human, a companion animal, or a livestock animal. The result may be any result achievable under the method including a DNA sequence or a ΔCt value.

The above and other objects may be achieved through the use of methods involving receiving a sample, isolating nucleic acid from the sample, adding a first oligonucleotide including a sequence selected from the group consisting of SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, and SEQ ID NO. 16 to a mixture comprising the nucleic acid; subjecting the mixture to conditions that allow nucleic acid amplification and identifying the sample as containing C. posadasii or not containing C. posadasii on the basis of a result of the nucleic acid amplification. If the first nucleic acid includes SEQ ID NO. 9 then the method may further comprise adding a second oligonucleotide including SEQ ID NO. 10 to the mixture. This aspect of the method may further comprise performing DNA sequencing on a product of the nucleic acid amplification or it may further comprise adding a third oligonucleotide including SEQ ID NO. 5 to the mixture. If the first nucleic acid includes SEQ ID NO. 11 then the method may further comprise adding a second oligonucleotide including SEQ ID NO. 12 to the mixture. This aspect of the method may further comprise performing DNA sequencing on a product of the nucleic acid amplification or it may further comprise adding a third oligonucleotide including SEQ ID NO. 6 to the mixture. If the first nucleic acid includes SEQ ID NO. 13 then the method may further comprise adding a second oligonucleotide including SEQ ID NO. 14 to the mixture. This aspect of the method may further comprise performing DNA sequencing on a product of the nucleic acid amplification or it may further comprise adding a third oligonucleotide including SEQ ID NO. 7 to the mixture. If the first nucleic acid includes SEQ ID NO. 15 then the method may further comprise adding a second oligonucleotide including SEQ ID NO. 16 to the mixture. This aspect of the method may further comprise performing DNA sequencing on a product of the nucleic acid amplification or it may further comprise adding a third oligonucleotide including SEQ ID NO. 8 to the mixture. In any of the above aspects, the third oligonucleotide may comprise a label. The label may be any label including a fluorescent label such as FAM, dR110, 5-FAM, 6FAM, dR6G, JOE, HEX, VIC, TET, dTAMRA, TAMRA, NED, dROX, PET, BHQ+, Gold540, or LIZ. The first oligonucleotide may be affixed to a solid substrate. The sample may comprise an environmental sample. In other aspects, the sample may be derived from a subject. A subject may be a human, a companion animal, or a livestock animal. The result may be any result achievable under the method including a DNA sequence or a ΔCt value.

The above and other objects may be achieved through the use of methods involving receiving a sample, isolating nucleic acid from the sample, adding a first oligonucleotide including a sequence selected from SEQ ID NO. 9, SEQ ID NO. 11, SEQ ID NO. 13, and SEQ ID NO. 15 to a mixture comprising the nucleic acid, adding a second nucleotide including a sequence selected from SEQ ID NO. 10, SEQ ID NO. 12, SEQ ID NO. 14, and SEQ ID NO. 16 to the mixture, subjecting the mixture to conditions that allow nucleic acid amplification, identifying the sample as containing C. immitis or not containing C. immitis based upon a result of the nucleic acid amplification, and identifying the sample as containing C. posadasii or not containing C. posadasii based upon a result of the nucleic acid amplification. The method may further comprise performing DNA sequencing upon a product of the nucleic acid amplification. The method may further comprise adding a third oligonucleotide including a sequence selected from the group consisting of SEQ ID NO. 1, SEQ ID NO. 2, SEQ ID NO. 3, and SEQ ID NO. 4 to the mixture and adding a fourth oligonucleotide including SEQ ID NO. 5, SEQ ID NO. 6, SEQ ID NO. 7, and SEQ ID NO. 8 to the mixture; wherein the third oligonucleotide comprises a first fluorescent label and the fourth oligonucleotide comprises a second fluorescent label and wherein the first fluorescent label and the second fluorescent label fluoresce at different wavelengths. If DNA sequencing is performed upon a product of the nucleic acid amplification, then the first oligonucleotide may include SEQ ID NO. 9 and the second oligonucleotide may include SEQ ID NO. 10. If a third oligonucleotide and a fourth oligonucleotide are used, then the first oligonucleotide may include SEQ ID NO. 9, the second oligonucleotide may include SEQ ID NO. 10, the third oligonucleotide may include SEQ ID NO. 1 and the fourth oligonucleotide may include SEQ ID NO. 5. If DNA sequencing is performed upon a product of the nucleic acid amplification, then the first oligonucleotide may include SEQ ID NO. 11 and the second oligonucleotide may include SEQ ID NO. 12. If a third oligonucleotide and a fourth oligonucleotide are used, then the first oligonucleotide may include SEQ ID NO. 11, the second oligonucleotide may include SEQ ID NO. 12, the third oligonucleotide may include SEQ ID NO. 2 and the fourth oligonucleotide may include SEQ ID NO. 6. If DNA sequencing is performed upon a product of the nucleic acid amplification, then the first oligonucleotide may include SEQ ID NO. 13 and the second oligonucleotide may include SEQ ID NO. 14. If a third oligonucleotide and a fourth oligonucleotide are used, then the first oligonucleotide may include SEQ ID NO. 13, the second oligonucleotide may include SEQ ID NO. 14, the third oligonucleotide may include SEQ ID NO. 3 and the fourth oligonucleotide may include SEQ ID NO. 7. If DNA sequencing is performed upon a product of the nucleic acid amplification, then the first oligonucleotide may include SEQ ID NO. 15 and the second oligonucleotide may include SEQ ID NO. 16. If a third oligonucleotide and a fourth oligonucleotide are used, then the first oligonucleotide may include SEQ ID NO. 15, the second oligonucleotide may include SEQ ID NO. 16, the third oligonucleotide may include SEQ ID NO. 4 and the fourth oligonucleotide may include SEQ ID NO. 8. The first fluorescent label and the second fluorescent label may be selected from the group consisting of FAM, dR110, 5-FAM, 6FAM, dR6G, JOE, HEX, VIC, TET, dTAMRA, TAMRA, NED, dROX, PET, BHQ+, Gold540, or LIZ. The first oligonucleotide may be affixed to a solid substrate. The sample may be any sample including an environmental sample or a sample from a subject. The subject may be any subject including a human, a companion animal, or a livestock animal. The subject may also be suspected of having a Coccidioides or other fungal infection. The result may be any result including a DNA sequence or a ΔCt value.

The above and other objects may be achieved through the use of kits involving a first oligonucleotide including a sequence selected from the group consisting of SEQ ID NO. 1, SEQ ID NO. 2, SEQ ID NO. 3, and SEQ ID NO. 4, a second oligonucleotide including a sequence selected from the group consisting of SEQ ID NO. 5, SEQ ID NO. 6, SEQ ID NO. 7, and SEQ ID NO. 8, and an indication of a result that signifies classification of a sample as containing a Coccidioides species selected from the group consisting of C. immitis and C. posadasii. If the first oligonucleotide includes SEQ ID NO. 1 and the second oligonucleotide includes SEQ ID NO. 5, then the kit may further comprise a third oligonucleotide including a sequence selected from the group consisting of SEQ ID NO. 9 and SEQ ID NO. 10. If the first oligonucleotide includes SEQ ID NO. 2 and the second oligonucleotide includes SEQ ID NO. 6, then the kit may further comprise a third oligonucleotide including a sequence selected from the group consisting of SEQ ID NO. 11 and SEQ ID NO. 12. If the first oligonucleotide includes SEQ ID NO. 3 and the second oligonucleotide includes SEQ ID NO. 7, then the kit may further comprise a third oligonucleotide including a sequence selected from the group consisting of SEQ ID NO. 13 and SEQ ID NO. 14. If the first oligonucleotide includes SEQ ID NO. 4 and the second oligonucleotide includes SEQ ID NO. 8, then the kit may further comprise a third oligonucleotide including a sequence selected from the group consisting of SEQ ID NO. 15 and SEQ ID NO. 16. The kit may further comprise an enzyme such as a DNA polymerase, including a thermostable DNA polymerase. The first oligonucleotide may be affixed to a substrate. The kit may further comprise a device to be used in collecting a sample. The result may comprise a ΔCt value. The result may comprise a nucleic acid sequence. The indication may comprise a positive control or a writing. A may be physically included in a kit or made available via a website. A writing may include an amplification plot. The indication may comprise software configured to detect the result as input and identification of the sample as containing

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 is a graphical depiction of assay performance over 358 isolates previously typed by microsatellite analysis.

FIG. 2 is a graphical depiction of assay specificity.

Elements and acts in the figures are illustrated for simplicity and have not necessarily been rendered according to any particular sequence or embodiment.

### DETAILED DESCRIPTION OF THE INVENTION

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. §112, ¶6. Thus, the use of the words “function,” “means” or “step” in the Detailed Description or Description of the Drawings or claims is not intended to somehow indicate a desire to invoke the special provisions of 35 U.S.C. §112, ¶6, to define the invention. To the contrary, if the provisions of 35 U.S.C. §112, ¶6 are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. §112, ¶6. Moreover, even if the provisions of 35 U.S.C. §112, ¶6 are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

In one embodiment of the invention, one or more probes identified as SEQ ID NO. 1, SEQ ID NO. 2, SEQ ID NO. 3, and SEQ ID NO. 4, alone or in combination are used to detect the presence or absence of one or more alleles specific to C. immitis, thereby detecting the presence of C. immitis in a sample. In another embodiment of the invention, one or more probes identified as SEQ ID NO. 5, SEQ ID NO. 6, SEQ ID NO. 7, and SEQ ID NO. 8 alone or in combination, are used to detect the presence of C. posadasii in a sample. In another embodiment of the invention, one or more probes identified as SEQ ID NO. 1, SEQ ID NO. 2, SEQ ID NO. 3, and SEQ ID NO. 4 are labeled with a first label and one or more probes identified as SEQ ID NO. 5, SEQ ID NO. 6, SEQ ID NO. 7, and SEQ ID NO. 8 are labeled with a second label that may be differentiated from the first label and used to differentiate whether or not the sample contains C. posadasii and/or C. immitis. In an additional embodiment, one or more oligonucleotides and other reagents that facilitate the detection of C. immitis, C. posadasii, or the differentiation of the two species may be incorporated into a kit.

Any compositions of matter disclosed herein are identified by a particular nucleic acid sequence. However, the invention contemplates any probe sequence similar enough to the listed sequence still able to bind sufficiently to the sample sequence that the conjugate sequence is identified, and/or the targeted polynucleotide is amplified through a nucleic acid amplification method. Therefore, the identifying sequence may include a mutation, truncation, or addition of extra nucleotides. A sequence having at least 60%, 70%, 80%, 90%, or 95% identity to the identifying sequence is still encompassed by the invention.

An allele includes any form of a particular nucleic acid that may be recognized as a form of the particular nucleic acid on account of its location, sequence, or any other characteristic that may identify it as being a form of the particular gene. Alleles include but need not be limited to forms of a gene that include point mutations, silent mutations, deletions, frameshift mutations, single nucleotide polymorphisms (SNPs), inversions, translocations, heterochromatic insertions, and differentially methylated sequences relative to a reference gene, whether alone or in combination. The presence or absence of an allele may be detected through the use of any process through which a specific nucleic acid molecule may be detected, including direct and indirect methods of detecting the presence or absence of an allele. An allele may occur in a non-coding or coding region of a genome. If it is in a coding region, it may affect a particular triplet codon. If the allele does affect the codon, it may change the amino acid in the protein resulting from expression of the allele. The exception is if the allele is a silent mutation. In that case, the allele is a mutation in the coding region that does not change the amino acid that the codon encodes. An allele may also be called a mutation or a mutant. An allele may be compared to another allele that may be termed a wild type form of an allele. In some cases, the wild type allele is more common than the mutant.

The invention may comprise methods detecting the presence of a particular fungus in a sample. A sample may be derived from anywhere that a fungus or any part of a fungus including fungal spores, buds, or hyphae may be found including soil, air, water, solid surfaces (whether natural or artificial,) culture media, foodstuffs, and any interfaces between or combinations of these elements. Additionally, a sample may be derived from a subject, such as a plant or animal, including humans. Samples derived from animals include but are not limited to biopsy or other in vivo or ex vivo analysis of prostate, breast, skin, muscle, facia, brain, endometrium, lung, head and neck, pancreas, small intestine, blood, liver, testes, ovaries, colon, skin, stomach, esophagus, spleen, lymph node, bone marrow, kidney, placenta, or fetus. Samples derived from subjects may also take the form of a fluid sample such as peripheral blood, lymph fluid, ascites, serous fluid, pleural effusion, sputum, bronchial wash, bronchioalveolar lavage fluid (BALF,) cerebrospinal fluid, semen, amniotic fluid, lacrimal fluid, stool, urine, hair, or any other source in which a fungus, or any part of a fungus might be present. Samples collected from a plant may be collected from part of a plant or from an entire plant. Samples may be collected by any method now known or yet to be disclosed, including swiping or swabbing an area or orifice, removal of a piece of tissue as in a biopsy, or any method known to collect bodily fluids. Samples may be suspected of containing a fungus if they are derived from a subject displaying symptoms of a fungal infection or from an environmental sample from an area in which a fungus is thought to be endemic.

Direct methods of detecting the presence of an allele include but are not limited to any form of DNA sequencing including Sanger, next generation sequencing, pyrosequencing, SOLID sequencing, massively parallel sequencing, pooled, and barcoded DNA sequencing or any other sequencing method now known or yet to be disclosed; PCR-based methods such as real-time PCR, quantitative PCR, reverse transcription PCR or any combination of these; allele specific ligation; comparative genomic hybridization; or any other method that allows the detection of a particular nucleic acid sequence within a sample or enables the differentiation of one nucleic acid from another nucleic acid that differs from the first nucleic acid by one or more nucleotides. A sample may be suspected of including a nucleic acid from a fungus of interest. Nucleic acids may include but need not be limited to RNA, cDNA, tRNA, mitochondrial DNA, plasmid DNA, siRNA, genomic DNA, or any other naturally occurring or artificial nucleic acid molecule. A subject may be any organism that may be infected by a fungus including plants, animals, chordates, mammals, humans, insects, endangered species, or any other organism of agricultural, environmental, or other significance.

In Sanger Sequencing, a single-stranded DNA template, a primer, a DNA polymerase, \nucleotides and a label such as a radioactive label conjugated with the nucleotide base or a fluorescent label conjugated to the primer, and one chain terminator base comprising a dideoxynucleotide (ddATP, ddGTP, ddCTP, or ddTTP, are added to each of four reaction (one reaction for each of the chain terminator bases). The sequence may be determined by electrophoresis of the resulting strands. In dye terminator sequencing, each of the chain termination bases is labeled with a fluorescent label of a different wavelength which allows the sequencing to be performed in a single reaction.

In pyrosequencing, the addition of a base to a single stranded template to be sequenced by a polymerase results in the release of a phyrophosphate upon nucleotide incorporation. An ATP sulfyrlase enzyme converts pyrophosphate into ATP which in turn catalyzes the conversion of luciferin to oxyluciferin which results in the generation of visible light that is then detected by a camera.

In SOLID sequencing, the molecule to be sequenced is fragmented and used to prepare a population of clonal magnetic beads (in which each bead is conjugated to a plurality of copies of a single fragment) with an adaptor sequence and alternatively a barcode sequence The beads are bound to a glass surface. Sequencing is then performed through 2-base encoding.

In massively parallel sequencing, randomly fragmented targeted DNA is attached to a surface. The fragments are extended and bridge amplified to create a flow cell with clusters, each with a plurality of copies of a single fragment sequence. The templates are sequenced by synthesizing the fragments in parallel. Bases are indicated by the release of a fluorescent dye correlating to the addition of the particular base to the fragment.

When a nucleic acid includes a particular sequence, the sequence may be a part of a longer nucleic acid or may be the entirety of the sequence. The nucleic acid may contain nucleotides 5′ of the sequence, 3′ of the sequence, or both. The concept of a nucleic acid including a particular sequence further encompasses nucleic acids that contain less than the full sequence that are still capable of specifically detecting an allele. Nucleic acid sequences may be identified by the IUAPC letter code which is as follows: A—Adenine base; C—Cytosine base; G—guanine base; T or U—thymine or uracil base. M-A or C; R-A or G; W-A or T; S-C or G; Y-C or T; K-G or T; V-A or C or G; H-A or C or T; D-A or G or T; B-C or G or T; N or X-A or C or G or T. Note that T or U may be used interchangeably depending on whether the nucleic acid is DNA or RNA. A sequence having less than 60% 70%, 80%, 90%, 95%, 99% or 100% identity to the identifying sequence may still be encompassed by the invention if it is able of binding to its complimentary sequence and/or facilitating nucleic acid amplification of a desired target sequence. If a sequence is represented in degenerate form; for example through the use of codes other than A, C, G, T, or U; the concept of a nucleic acid including the sequence also encompasses a mixture of nucleic acids of different sequences that still meet the conditions imposed by the degenerate sequence.

Indirect methods of detecting an allele generally involve assessing the expression of material created from a genomic DNA template such as a RNA or protein molecule. Such expression may be assessed by any of a number of methods used currently in the art and yet to be developed. Examples include any nucleic acid detection method including the following nonlimiting examples, microarray analysis, RNA in situ hybridization, RNAse protection assay, Northern blot, RTPCR, and quantitative PCR, and QRTPCR. Other examples include any process of detecting expression that uses an antibody including the following nonlimiting examples, flow cytometry, immunohistochemistry, ELISA, Western blot, Northwestern blot, and immunoaffinity chromatograpy. Antibodies may be monoclonal, polyclonal, or any antibody fragment including an Fab, F(ab)₂, Fv, scFv, phage display antibody, peptibody, multispecific ligand, or any other reagent with specific binding to a target. Other methods of assessing protein expression include the following nonlimiting examples: HPLC, mass spectrometry, protein microarray analysis, PAGE analysis, isoelectric focusing, 2-D gel electrophoresis, and enzymatic assays.

A nucleic acid may be added to a sample by any of a number of methods including manual methods, mechanical methods, or any combination thereof. The presence of the allele may be signified by any of a number of methods including amplification of a specific nucleic acid sequence, sequencing of a native or amplified nucleic acid, or the detection of a label either bound to or released from the nucleic acid. Addition of the nucleic acid to the sample also encompasses addition of the nucleic acid to a sample in which the target allele to which the nucleic acid has specificity is absent.

In some aspects of the invention, the presence of an allele may be established by binding to a microarray such as a DNA chip. Examples of DNA chips include chips in which a number of single stranded oligonucleotide probes are affixed to a solid substrate such as silicon glass. Oligonucleotides with a sequence complementary to an allele are capable of specifically binding to that allele to the exclusion of alleles that differ from the specific allele by one or more nucleotides. Labeled sample DNA is hybridized to the oligonucleotides and detection of the label is correlated with binding of the sample and consequently the presence of the allele in the sample.

In allele-specific hybridization, oligonucleotide sequences representing all possible variations at a polymorphic site are included on a chip. The chip and sample are subject to conditions under which the labeled sample DNA will bind only to an oligonucleotide with an exact sequence match. In allele-specific primer extension, sample DNA hybridized to the chip may be used as a synthesis template with the affixed oligonucleotide as a primer. Under this method, only the added dNTP's are labeled. Incorporation of the labeled dNTP then serves as the signal indicating the presence of the allele. The fluorescent label may be detected by any of a number of instruments configured to read at least four different fluorescent labels on a DNA chip. In another alternative, the identity of the final dNTP added to the oligonucleotide may be assessed by mass spectrometry. In this alternative, the dNTP's may, but need not be labeled with a label of known molecular weight.

A nucleic acid probe may be affixed to a substrate. In other aspects of the invention, a sample may be affixed to the substrate. A probe or sample may be covalently bound to the substrate or it may be bound by some non covalent interaction including electrostatic, hydrophobic, hydrogen bonding, Van Der Waals, magnetic, or any other interaction by which a probe such as an oligonucleotide probe may be attached to a substrate while maintaining its ability to recognize the allele to which it has specificity. A substrate may be any solid or semi solid material onto which a probe may be affixed, attached or printed, either singly or in the presence of one or more additional probes or samples as is exemplified in a microarray. Examples of substrate materials include but are not limited to polyvinyl, polysterene, polypropylene, polyester or any other plastic, glass, silicon dioxide or other silanes, hydrogels, gold, platinum, microbeads, micelles and other lipid formations, nitrocellulose, or nylon membranes. The substrate may take any form, including a spherical bead or flat surface. For example, the probe may be bound to a substrate in the case of an array or an in situ PCR reaction. The sample may be bound to a substrate in the case of a Southern Blot.

A nucleic acid probe may include a label. A label may be any substance capable of aiding a machine, detector, sensor, device, or enhanced or unenhanced human eye from differentiating a labeled composition from an unlabeled composition. Examples of labels include but are not limited to: a radioactive isotope or chelate thereof, dye (fluorescent or nonfluorescent,) stain, enzyme, or nonradioactive metal. Specific examples include but are not limited to: fluorescein, biotin, digoxigenin, alkaline phosphatese, biotin, streptavidin, ³H, ¹⁴C, ³²P, ³⁵S, or any other compound capable of emitting radiation, rhodamine, 4-(4′-dimethylamino-phenylazo)benzoic acid (“Dabcyl”); 4-(4′-dimethylamino-phenylazo)sulfonic acid (sulfonyl chloride) (“Dabsyl”); 5-((2-aminoethyl)-amino)-naphtalene-1-sulfonic acid (“EDANS”); Psoralene derivatives, haptens, cyanines, acridines, fluorescent rhodol derivatives, cholesterol derivatives; ethylenediaminetetraaceticacid (“EDTA”) and derivatives thereof or any other compound that may be differentially detected. The label may also include one or more fluorescent dyes optimized for use in genotyping. Examples of such dyes include but are not limited to: dR110, 5-FAM, 6FAM, dR6G, JOE, HEX, VIC, TET, dTAMRA, TAMRA, NED, dROX, PET, and LIZ.

In some aspects of the invention, the allele may be detected by quantitative PCR analysis, which may be performed using a kit containing components that facilitate genotyping analysis. Genotyping analysis may be performed using a probe that is capable of hybridizing to a nucleic acid sequence of interest. Probes may include nucleic acids, oligonucleotides (DNA, or RNA), proteins, protein complexes, conjugates, natural ligands, small molecules, nanoparticles, or any any combination of molecules that includes one or more of the above, or any other molecular entity capable of specific binding to a any allele, whether such molecular entity exists now or is yet to be disclosed. In one aspect of the invention, the probe comprises an oligonucleotide. The concept of oligonucleotides includes any DNA or RNA molecule of two or more nucleotides, whether from a natural source, artificially synthesized, or produced through the use of recombinant DNA technology. A nucleotide is an individual deoxyribonucleotide or ribonucleotide base. Examples of nucleotides include but are not limited to: adenine, thymine, guanine, cytosine, and uracil which may be abbreviated as A, T, G, C, or U in representations of oligonucleotide sequence. The length of the oligonucleotide depends on how the oligonucleotide will be used. One skilled in the art would understand the approximate length of oligonucleotide necessary in any given method. Depending on the method, an oligonucleotide may be 0 to 1000 bases in length. In other aspects, it may be 5 to 500 bases in length, 5 to 100 bases in length, 5 to 50 bases in length, or 10 to 30 bases in length.

Nucleic acids that may be subjected to amplification may be from any source. In general, nucleic acid amplification is a process by which copies of a nucleic acid may be made from a source nucleic acid. In some nucleic amplification methods, the copies are generated exponentially. Examples of nucleic acid amplification include but are not limited to: the polymerase chain reaction (PCR), ligase chain reaction (LCR,) self-sustained sequence replication (3SR), nucleic acid sequence based amplification (NASBA,) strand displacement amplification (SDA,) amplification with Qβ replicase, whole genome amplification with enzymes such as φ29, whole genome PCR, in vitro transcription with Klenow or any other RNA polymerase, or any other method by which copies of a desired sequence are generated.

Polymerase chain reaction (PCR) is a particular method of amplifying DNA, generally involving the mixing of a nucleic sample, two or more primers, a DNA polymerase, which may be a thermostable DNA polymerase such as Taq or Pfu, and deoxyribose nucleoside triphosphates (dNTP's). In general, the reaction mixture is subjected to temperature cycles comprising a denaturation stage, (typically 80-100° C.) an annealing stage with a temperature that may based on the melting temperature (Tm) of the primers and the degeneracy of the primers, and an extension stage (for example 40-75° C.) In real-time PCR analysis, additional reagents, methods, optical detection systems, and devices are used that allow a measurement of the magnitude of fluorescence in proportion to concentration of amplified DNA. In such analyses, incorporation of fluorescent dye into the amplified strands may be detected or labeled probes that bind to a specific sequence during the annealing phase release their fluorescent tags during the extension phase. Either of these will allow a quantification of the amount of specific DNA present in the initial sample. RNA may be detected by PCR analysis by creating a DNA template from RNA through a reverse transcriptase enzyme.

The invention may be used in the identification of fungus. Examples of fungi that may be identified or ruled out using the invention include but need not be limited to: pathogenic fungi such as Candida quercitrusa, Absidia corymbifera, Acremonium strictum, Aspergillus flavus, Aspergillus fumigatus, Aspergillus nidulans, Aspergillus niger, Aspergillus versicolor, Aureobasidium pullulans, Candida albicans, Candida famata, Candida guilliermondii, Candida haemulonii, Candida intermedia, Candida lusitaniae, Candida pararugosa, Candida rugosa, Candida tropicalis, Chaetomium globosum, Coccidioides immitis, Coccidioides posadasii, Corynespora cassiicola, Cryptococcus neoformans, Cunnighamella bertholletiae, Epidermophyton floccosum, Exophiala dermatitidis, Fonsecaea pedrosoi, Fusarium equiseti, Fusarium oxysporum, Fusarium solani, Geotrichum candidum, Geotrichum capitatum, Malassezia furfur, Microsporum canis, Microsporum gypseum, Neurospora crassa, Paecilomyces lilacinus, Paecilomyces sinensis, Paecilomyces variotii, Penicillium marneffei, Pichia ohmeri, Rhizopus microsporus, Rhizopus oryzae, Rhodotorula minuta, Rhodotorula mucilaginosa, Rhodotorula slooffiae, Saccharomycopsis crataegensis, Scedosporium apiospermum, Scedosporium prolificans, Sporothrix schenckii, Stephanoascus ciferrii, Trichophyton mentagrophytes, Trichophyton rubrum, Trichosporon asahii, Trichosporon asteroides, Trichosporon cutaneum, Trichosporon dermatis, Trichosporon faecale, Trichosporon montevideense, Trichosporon mucoides, and Trichosporon ovoides as well as environmental fungi such as Gigaspora gigantea, Acaulospora “brown”, Hebeloma crustuliniformae, Comprinus micaceous, Sarcosphaera crassa, Pholiota destruens, Pleurotus ostreatus, Cortinarius sp., Helvella queletii, Sclerogaster xerophila, Melanogaster magnisporas, Sedecula pulvinata, Elaphomyces decipiens, Clavulina cristata, Rizopogon sp, Hebeloma crustuliniformae, Tricholoma polulinum, Lactarius sp., Cortinarius sp., Agaricus sp., Xanthomendoza galericulata, Endoconidioma sp., Cladosporium cladosporioides, Phoma sp., Cytospora sp., and Alternaria sp.

Kits that facilitate nucleic acid based methods may further include one or more of the following: specific nucleic acids such as oligonucleotides, labeling reagents, enzymes including PCR amplification reagents such as the DNA polymerases Taq or Pfu, reverse transcriptase, or one or more other polymerases, and/or reagents that facilitate hybridization. Specific nucleic acids may include nucleic acids, polynucleotides, oligonucleotides (DNA, or RNA), or any combination of molecules that includes one or more of the above, or any other molecular entity capable of specific binding to a nucleic acid marker. In one aspect of the invention, the specific nucleic acid comprises one or more oligonucleotides capable of hybridizing to the marker.

A specific nucleic acid may include a label. A label may be any substance capable of aiding a machine, detector, sensor, device, or enhanced or unenhanced human eye from differentiating a sample that that displays positive expression from a sample that displays reduced expression. Examples of labels include but are not limited to: a radioactive isotope or chelate thereof, a dye (fluorescent or nonfluorescent,) stain, enzyme, or nonradioactive metal. Specific examples include but are not limited to: fluorescein, biotin, digoxigenin, alkaline phosphatase, biotin, streptavidin, ³H, ¹⁴C, ³²P, ³⁵S, or any other compound capable of emitting radiation, rhodamine, 4-(4′-dimethylaminophenylazo) benzoic acid (“Dabcyl”); 4-(4′-dimethylamino-phenylazo)sulfonic acid (sulfonyl chloride) (“Dabsyl”); 4-(4-aminoethyl)-amino)-naphtalene-1-sulfonic acid (“EDANS”); Psoralene derivatives, haptens, cyanines, acridines, fluorescent rhodol derivatives, cholesterol derivatives; ethylene diamine tetra-acetic acid (“EDTA”) and derivatives thereof or any other compound that signals the presence of the labeled nucleic acid. In one embodiment of the invention, the label includes one or more dyes optimized for use in genotyping. Examples of such dyes include but are not limited to: dR110, 5-FAM, 6FAM, dR6G, JOE, HEX, VIC, TET, dTAMRA, TAMRA, NED, dROX, PET, and LIZ.

An oligonucleotide may be any polynucleotide of at least 2 nucleotides. Oligonucleotides may be less than 10, less than 15, less than 20, less than 30, less than 40, less than 50, less than 75, less than 100, less than 200, or less than 500 nucleotides in length. While oligonucleotides are often linear, they may, depending on their sequence and conditions, assume a two- or three-dimensional structure. Oligonucleotides may be chemically synthesized by any of a number of methods including sequential synthesis, solid phase synthesis, or any other synthesis method now known or yet to be disclosed. Alternatively, oligonucleotides may be produced by recombinant DNA based methods. In some aspects of the invention, an oligonucleotide may be 2 to 1000 bases in length. In other aspects, it may be 5 to 500 bases in length, 5 to 100 bases in length, 5 to 50 bases in length, or 10 to 30 bases in length. One skilled in the art would understand the length of oligonucleotide necessary to perform a particular task. Oligonucleotides may be directly labeled, used as primers in PCR or sequencing reactions, or bound directly to a solid substrate as in oligonucleotide arrays.

A nucleotide is an individual deoxyribonucleotide or ribonucleotide base. Examples of nucleotides include but are not limited to: adenine, thymine, guanine, cytosine, and uracil, which may be abbreviated as A, T, G, C, or U in representations of oligonucleotide or polynucleotide sequence. Any molecule of two or more nucleotide bases, whether DNA or RNA, may be termed a nucleic acid.

A nucleic acid reagent used to detect to an allele may be affixed to a solid substrate. Alternatively, the sample may be affixed to a solid substrate and the nucleic acid reagent placed into a mixture. For example, the nucleic acid reagent may be bound to a substrate in the case of an array or the sample may be bound to a substrate as the case of a Southern Blot, Northern blot or other method that affixes the sample to a substrate. A nucleic acid reagent or sample may be covalently bound to the substrate or it may be bound by some non covalent interaction including electrostatic, hydrophobic, hydrogen bonding, Van Der Waals, magnetic, or any other interaction by which a probe such as an oligonucleotide probe may be attached to a substrate while maintaining its ability to recognize the allele to which it has specificity. A substrate may be any solid or semi solid material onto which a probe may be affixed, attached or printed, either singly or in the formation of a microarray. Examples of substrate materials include but are not limited to polyvinyl, polysterene, polypropylene, polyester or any other plastic, glass, silicon dioxide or other silanes, hydrogels, gold, platinum, microbeads, micelles and other lipid formations, nitrocellulose, or nylon membranes. The substrate may take any shape, including a spherical bead or flat surface.

A kit may also contain an indication that links the output of the kit to a particular result. For example, an indication may be one or more sequences or that signify the identification of a particular fungal phylum, class, order, family, genus species, subspecies, strain or any other delineation of a group of fungi. A kit may contain a standard curve configured to quantify the amount of fungus present in a sample. An indication includes any guide that links the output of the kit to a particular result. The indication may be a particular sequence, a particular ΔCt level in a qRTPCR reaction, a level of fluorescence or radioactive decay, a value derived from a standard curve, or from a control, or any combination of these and other outputs. The indication may be printed on a writing that may be included in the kit or it may be posted on the Internet or embedded in a software package.

### EXAMPLE

The Inventors have developed a PCR allelic-discrimination assay that accurately distinguishes between C. immitis and C. posadasii based upon the presence of one or more single-nucleotide polymorphisms (SNPs). The invention targets canonical SNPs (canSNPs). CanSNPs are typically synonymous mutations and are, therefore, conserved characters that mark pivotal evolutionary points found in related phylogenetic groupings (See Reference 12). A single canSNP is all that is required to identify a particular species, strain, or clone. The Inventors identified several canSNPs that separate two Coccidioides species and created compositions of matter and methods that allow rapid differentiation of Coccidioides species in epidemiological and other research studies. Further, identification of a particular Coccidioides species will result in improved prediction of clinical outcome as well as differential treatment options.

In order to identify SNPs that might differentiate C. immitis and C. posadasii, whole genome sequences from fourteen isolates (See Reference 10) (four C. immitis and ten C. posadasii) were compared. Perl and Java scripts based on MUMmer 3.20 (See Reference 11) was used to discover SNPs between all strain pairs of C. immitis (strains RS, H538.4, RMSCC 2394, RMSCC 3703) and C. posadasii (strains C735, CPA 0001, CPA 0020, CPA 0066, RMSCC 1037, RMSCC 1038, RMSCC 1040, RMSCC 2133, RMSCC 3488, RMSCC 3700, Silveira). In order to be included in the analysis, a SNP was required to be from a region shared across all taxa in the comparisons. Because so few SNP loci were shared between species, SNPs were also compared within C. immitis, exclusive of C. posadasii, and vice-versa. Eight species-discriminating SNPs were identified and assays were designed around four of these, using Primer Express version 3.0 software (Applied Biosystems, Foster City, Calif.). One probe was designed to hybridize to a sequence from C. immitis 5′ATTGTCCAGTATGAGGAT-3′ (SEQ ID NO. 1), and the other to hybridize to the C. posadasii sequence 5′-ATTGTCCAGAATGAGGAT-3′ (SEQ ID NO 2). The probe of SEQ ID NO. 1 was conjugated with the fluorescent dye FAM and the probe of SEQ ID NO. 2 was conjugated with the fluorescent dye VIC.

All assays were initially screened across a geographically diverse panel consisting of seven C. immitis and nine C. posadasii strains originally isolated from varying specimen types (Table 1).

TABLE 1
Screening panel isolates (1, 4)

| Strain | | | Isolate | Year |
|---|---|---|---|---|
| ID | Species | Place of Origin | Type | Collected |
| RS | C. immitis | vaccine strain - | Clinical | 1950 |
| | | origin unknown | | |
| 2394 | C. immitis | San Diego, CA | Clinical | 2001* |
| Silveira | C. posadasii | San Joaquin | Clinical | 1953 |
| | | Valley, CA | | |
| 3703 | C. immitis | San Diego, CA | Clinical | 2001* |
| 3488 | C. posadasii | Sonora, N. MEX | Clinical | 2001* |
| CPA0066 | C. posadasii | Tucson, AZ | Environ- | 2004 |
| | | | mental | |
| VFC040 | C. immitis | Tuscon, AZ | Clinical | 1994 |
| 3377 | C. immitis | Monterey, CA | Clinical | 2001* |
| 3476b | C. immitis | Michoacan, S. MEX | Clinical | 2001* |
| 3505 | C. immitis | Coahuila, N. MEX | Clinical | 2001* |
| VFC057 | C. posadasii | Zurich, SUI | Clinical | 2000 |
| 3187 | C. posadasii | Tuscon, AZ | Clinical | 1991 |
| 3198 | C. posadasii | Tuscon, AZ | Clinical | 1990 |
| 3395 | C. posadasii | Tuscon, AZ | Clinical | 1981 |
| 2008 | C. posadasii | Bakersfield, CA | Clinical | 2007* |
| VFC047 | C. posadasii | Colorado Springs, | Clinical | 1997 |
| | | CO | | |

*indicates year referenced; year collected unknown

This panel (n=16) was screened in 10.0-4 reaction volumes and all additional reactions were carried out in 5.0-μL reaction volumes containing 900 nM of a forward primer of sequence 5′-CGTGTGGCCTTGCAGTATAGC-3′ (SEQ ID NO. 9) and 900 nM of a reverse primer of sequence 5′-TTTACGCCGTAGCCTTTGATG-3′ (SEQ ID NO. 10) 125 nM of each probe, 1× Applied Biosystems Genotyping Master Mix and 1.0 μL of sample. Thermal cycling was performed using the ABI 7900HT sequence detection system under the following conditions: 50° C. for 2 min, 95° C. for 10 min, and 40 cycles of 95° C. for 15 s and 58° C. for 1 min.

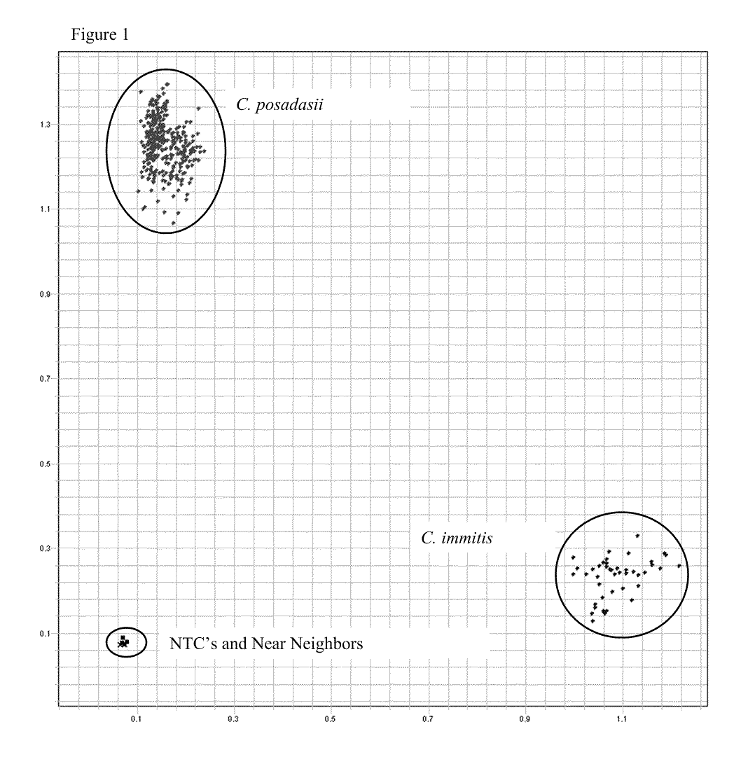

Upon completion of initial screening, one selected assay was used to genotype a collection of 358 C. immitis and C. posadasii human, animal, and environmental isolates from a broad geographic range. This assay (CocciDiff) demonstrated 99.7% specificity differentiating all C. immitis isolates from all C. posadasii isolates (FIG. 1). Specimen DNA was arrayed in reaction plates randomly and testing was blinded to limit technician bias. Isolate species identification was confirmed by microsatellite analysis previously conducted in the laboratories of origin. One isolate in the collection was previously typed as C. immitis according to microsatellite analysis. However, limited epidemiological data available for this sample indicates that the original clinical sample was collected from a patient at a Maricopa County, Arizona hospital, possibly indicating that this sample was previously typed incorrectly. All isolates were screened in triplicate and demonstrated 100% concordance. Data points along the x axis represent isolates possessing the C. immitis-specific allele (n=43). Data points along they axis represent isolates possessing the C. posadasii-specific allele (n=315). Black data points near the plot origin are negative no-template controls (NTCs; n=2) and genetic near-neighbor isolates (n=2).

To further validate assay specificity, we ran the CocciDiff assay against a screening panel consisting of eighteen near-neighbor isolates, differential diagnostic isolates and other non-target controls (Table 2).

TABLE 2
members of screening panel

| | |
|---|---|
| Differential Diagnostic | Streptococcus pneumonia |
| Isolates | Strep. viridans |
| | Candida albicans |
| | Acinetobacter baumanni |
| | Pseudomonas aeruginosa |
| | Escherichia coli |
| | Staphylococcus aureus - methicillin resistant |
| | Staph. aureus - methicillin susceptible |
| | Staph. haemolyticus |
| | Klebsiella pneumoniae |
| | Mycoplasma pneumoniae |
| | Enterococcus faecium |
| | Moraxella catarrhalis |
| | Haemophilus influenzae |
| Near-Neighbor and | Uncinocarpus reesii |
| Background Isolates | Histoplama capsulatum |
| | Homo sapiens |

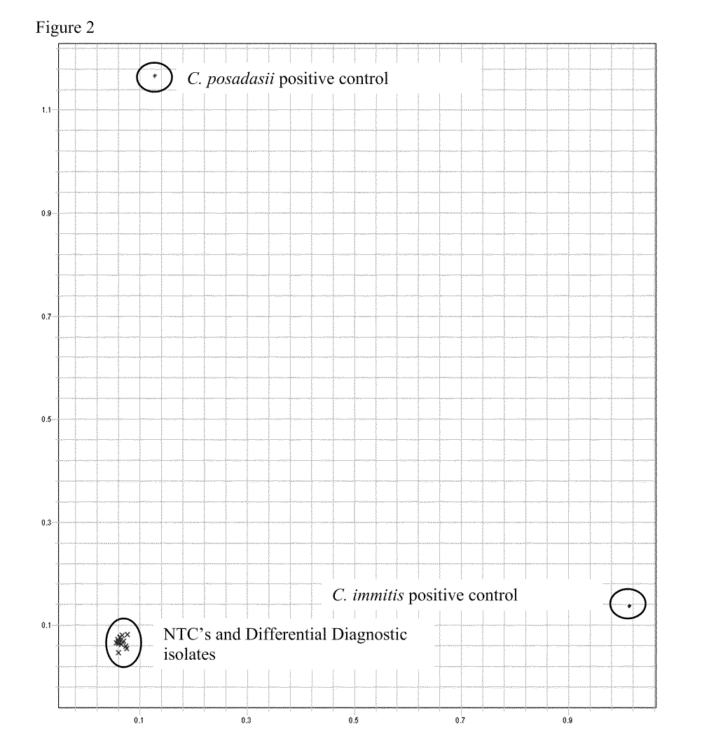

All non-target isolates screened across the CocciDiff assay failed to amplify (FIG. 2). A ten-fold dilution series from 30 ng to 3 fg was prepared for a single C. immitis and C. posadasii isolate and ran these across CocciDiff to determine assay detection threshold. The CocciDiff routine detection threshold is 10 fungal genomic copies, with 1 fungal genomic copy being sporadically detectable. The lone data point along the x-axis near the right side of the graph is a C. immitis positive control (n=1). The data point along the y-axis near the top of the graph is a C. posadasii positive control (n=1). All points near the plot origin are NTCs (n=2) and differentially diagnostic and background isolates (n=16). All NTCs, differentially diagnostic, and background isolates failed to amplify.

Further validation of the CocciDiff assay was performed with regard to using the assay as a potential diagnostic tool with actual clinical samples. CocciDiff was used to screen 665 DNAs to test for sensitivity. The assay was also used to test against additional 41 non-target species (Table 3).

TABLE 3
Additional species screened using CocciDiff assay.

| | Total |
|---|---|
| Species | screened |
| Abiotrophia granulicattella | 1 |
| Acinetobacter baumanni | 1 |
| Acromobacter xylosoxidans | 1 |
| Bacillus spp. | 2 |
| Bacteroides fragilis | 1 |
| Bacteroides uniformis | 1 |
| Bordetella bronchiseptica | 1 |
| Burkholderia cepacia | 1 |
| Burkholderia psuedomallei | 3 |
| Candida albicans | 1 |
| Candida glabrata | 2 |
| Candida parapsilosis | 3 |
| Candida tropicalis | 1 |
| Chryseobacterium indologenes | 1 |
| Corynebacterium spp. | 1 |
| Coxiella burnetii | 2 |
| Enterobacter aerogenes | 2 |
| Enterobacter cloacae | 9 |
| Enterococcus faecium | 2 |
| Enterococcus sp | 2 |
| Francisella tulerensis | 2 |
| Group F Streptococcus | 1 |
| Haemophilus parainfluenza | 2 |
| Human gDNA | 2 |
| Legionella pneumophila | 1 |
| Listeria monocytogenes | 1 |
| Micrococcus spp. | 1 |
| Neisseria meningitidis | 3 |
| Neisseriea gonorrhoeae | 3 |
| Propionibacterium spp. | 1 |
| Providencia stuartii | 1 |
| Staphylococcus aureus | 4 |
| Staphylococcus epidermidis | 5 |
| Staphylococcus saprophyticus | 1 |
| Streptococcus agalactiae | 1 |
| Streptococcus mitis | 1 |
| Streptococcus pnuemoniae | 4 |
| Streptococcus pyogenes | 3 |
| Streptococcus salivarius | 1 |
| Streptococcus viridans | 3 |
| Unspeciated-CNS | 6 |
| Vancomycin Resistant Enterococcus | 8 |
| Vancomycin Resistant Staph | 6 |
| Vancomycin Sensitive Enterococcus | 2 |

All were negative on CocciDiff assay.

Clinical Validation:

Twenty-five respiratory samples (BAL and sputa) were tested using the CocciDiff assay. When comparing subsequent PCR results to the clinical microbiology results on samples from the same patients, we found concordance between culture and CocciDiff for all four patients with a positive culture result for unspecified Coccidioides. Seventeen of the twenty samples negative by culture were also negative using the assay. Three samples that were not positive by culture were found positive by CocciDif. In the case of sample TG11448, a patient's respiratory sample was culture negative in the clinical lab, but a subsequent tissue biopsy was culture positive. The CocciDiff PCR assay for this sample was negative. (Table 4).

TABLE 4
Coccidiff assay in clinical specimens.

| | | | Cocci | |
|---|---|---|---|---|
| Specimen | Respiratory | Cocci DNA | infection | |
| Type | Culture Result | Probe | status | CocciDiff result |
| BAL | NFO | NA | NA | UD |
| Sputum | NFO | NA | NA | UD |
| Sputum | NFO | NA | NA | UD |
| Sputum | NFO | NA | NA | UD |
| Sputum | NFO | NA | NA | UD |
| Sputum | NFO | NA | NA | UD |
| BAL | NFO | POS | Active | C. posadasii |
| BAL | NFO | NA | NA | C. posadasii |
| BAL | NFO | NA | NA | UD |
| BAL | NG | NA | NA | UD |
| Sputum | NFO | NA | NA | UD |
| BAL | NFO | NA | NA | UD |
| BAL | ACBA | NA | NA | UD |
| BAL | NG | NA | NA | UD |
| BAL | NFO | NA | NA | UD |
| BAL | NG | NA | NA | C. posadasii |
| BAL | NFO | POS | Active | C. posadasii |
| Sputum | NFO | NA | NA | UD |
| BAL | NG | NA | NA | C. posadasii |
| BAL | NFO | POS | Active | C. posadasii |
| BAL | NG | NA | Dormant | UD |
| Tissue bx | NG | Node POS | | |
| BAL | NG | POS | Active | C. posadasii |
| BAL | NFO | NA | NA | UD |
| Sputum | Cocci/ASSP | POS | Active | C. posadasii |
| Sputum | Not Cultured | NA | NA | UD |

Key: For Respiratory Culture Result, NFO = normal flora only. NG = no growth. ACBA = Acentiobacter baumanii. ASSP = Aspergillus sp. The Cocci DNA probe is a test by Accuprobe (See Reference 18) currently used to detect Coccidioides immitis isolated from culture. When no fungus grows in culture, the Cocci DNA probe cannot be used, so for NFO and NG, the Cocci DNA probe is necessarily not applicable (NA). UD = undetermined because of a lack of nucleic amplification product.

TABLE 5
Sequence Identifications
Probes:

| Sequence | | |
|---|---|---|
| Identi- | | |
| fication | Sequence | Organism |
| SEQ ID | ATTGTCCAGTATGAGGAT | C. immitis |
| NO. 1 | | |
| SEQ ID | AACATCAAGACCTCC | C. immitis |
| NO. 2 | | |
| SEQ ID | AACGTTGAGATAATTACAGGT | C. immitis |
| NO. 3 | | |
| SEQ ID | ACGCCAATCAGAGCA | C. immitis |
| NO. 4 | | |
| SEQ ID | ATTGTCCAGAATGAGGAT | C. posadasii |
| NO. 5 | | |
| SEQ ID | CATCAAGGCCTCC | C. posadasii |
| NO. 6 | | |
| SEQ ID | ACGTTGAGATAATTACGGGT | C. posadasii |
| NO. 7 | | |
| SEQ ID | ACGCCAATAAGAGCA | C. posadasii |
| NO. 8 | | |

Primers:

| Sequence | | | Used |
|---|---|---|---|
| Identi- | | Orien- | with |
| fication | Sequence | tation | probes: |
| SEQ ID | CGTGTGGCCTTGCAGTATAGC | Forward | SEQ ID |
| NO. 9 | | | NO. 1 |
| SEQ ID | TTTACGCCGTAGCCTTTGATG | Reverse | SEQ ID |
| NO. 10 | | | NO. 5 |
| SEQ ID | AGGCCTTCTGCTTGGAATCC | Forward | SEQ ID |
| NO. 11 | | | NO. 2 |
| SEQ ID | ATATTCCGCAATTTCAAGGAGAAG | Reverse | SEQ ID |
| NO. 12 | | | NO. 6 |
| SEQ ID | GGATACGTCGCTTTCCAACTCT | Forward | SEQ ID |
| NO. 13 | | | NO. 3 |
| SEQ ID | CGCTCTATGTGATCGACACTTGA | Reverse | SEQ ID |
| NO. 14 | | | NO. 7 |
| SEQ ID | ACGTGTCCGGCTATTTGCAT | Forward | SEQ ID |
| NO. 15 | | | NO. 4 |
| SEQ ID | CAGCTGTCGCGGAATAACCT | Reverse | SEQ ID |
| NO. 16 | | | NO. 8 |

### REFERENCES

So as to reduce the complexity and length of the Detailed Specification, and to fully establish the state of the art in certain areas of technology, Inventors herein expressly incorporates by reference all of the following materials identified in each numbered paragraph below.
1. Mandel M A et al, Eukaryot Cell 6, 1189-1199 (2007).
2. Cox R A and Magee D M, Clin Microbiol Rev 17, 804-839 (2004).
3. Rippon J W, Medical Mycology: the Pathogenic Fungi and Pathogenic Actinomycetes 3^{rd} ed., WB Saunders, Philadelphia (1988).
4. Fisher M C et al, Mycologia 94, 73-84 (2002).
5. Arizona Department of Health Services, Rate of reported cases of notifiable diseases by year for Arizona, 1997-2007, per 100,000 population (accessed 2008 Oct. 3) http://www.azdhs.gov/phs/oids/pdf/rates1997_{—}2007.pdf.
6. Ampel N M et al, Clin Infect Dis 27, 1528-1530 (1998).
7. Galgiani J, Ann Intern Med 130, 293-300 (1999).
8. Binnicker M J et al, J Clin Micro 45, 173-178 (2007).
9. Jewell K et al, Med Mycol 46, 449-55 (August, 2008).
10. Coccidioides group Database [database on the Internet]. Cambridge (MA): Broad Institute of MIT and Harvard. C2008—[cited 2008 Oct. 3]. Available from: http://www.broad.mit.edu/annotation/genome/coccidioides_group/MultiHome.html.
11. Kurtz S, et al. Genome Biol 5, R12 (2004).
12. Keim P et al, Infect Genet Evol 4, 205-213 (2004).
13. Milliman U.S. Pat. No. 5,284,747 filed 8 Dec. 1991.
14. Milliman, U.S. Pat. No. 5,501,951, filed 6 Jan. 1994.
15. Cole et al, US Patent Application No. 2003/0224013, filed 18 Apr. 2003.
16. TaqMan Genotyping Master Mix Protocol, Part Number 4371131 Rev. A, Applied Biosystems, February, 2007.
17. ABI Prism® 7900HT Sequence Detection System User's Manual, Part Number 4317596, Applied Biosystems.
18. GenProbe AccuProbe Coccidioides Immitis Culture Identification Test package insert. GEN-PROBE INCORPORATED, San Diego, Calif. 92121 USA.© 01992, 1998, 1999, 2001 Gen-Probe Incorporated

We claim:

1. A method of determining the presence or absence of C. immitis in a sample, comprising:
receiving the sample and isolating nucleic acid from the sample,
adding a first oligonucleotide including a sequence selected from the group consisting of SEQ ID NO. 1, SEQ ID NO. 2, SEQ ID NO. 3, SEQ ID NO. 4, SEQ ID NO. 9, SEQ ID NO. 10, SEQ ID NO. 11, SEQ ID NO. 12, SEQ ID NO. 13, SEQ ID NO. 14, SEQ ID NO. 15, and SEQ ID NO. 16 to a mixture comprising the nucleic acid;
subjecting the mixture to conditions that allow nucleic acid amplification; and
identifying the sample as containing C. immitis or not containing C. immitis on the basis of a result of the nucleic acid amplification.

2. The method of claim 1 wherein the first oligonucleotide includes SEQ ID NO. 9, further comprising adding a second oligonucleotide including SEQ ID NO. 10 to the mixture.

3. The method of claim 2 further comprising performing DNA sequencing on a product of the nucleic acid amplification.

4. The method of claim 2 further comprising and adding a third oligonucleotide including SEQ ID NO. 1 to the mixture.

5. The method of claim 1, wherein the first oligonucleotide includes SEQ ID NO. 11, further comprising adding a second nucleotide including SEQ ID NO. 12 to the mixture.

6. The method of claim 5 further comprising performing DNA sequencing on a product of the nucleic acid amplification.

7. The method of claim 5 further comprising adding a third oligonucleoide including SEQ ID NO. 2 to the mixture.

8. The method of claim 1 wherein the first oligonucleotide includes SEQ ID NO. 13, further comprising adding a second oligonucleotide including SEQ ID NO. 14 to the mixture

9. The method of claim 8 further comprising performing DNA sequencing on a product of the nucleic acid amplification.

10. The method of claim 8 further comprising adding a third oligonucleotide including SEQ ID NO. 3 to the mixture.

11. The method of claim 1 wherein the first oligonucleotide includes SEQ ID NO. 15 further comprising adding a second oligonucleotide including SEQ ID NO. 16.

12. The method of claim 11 further comprising performing DNA sequencing on a product of the nucleic acid amplification.

13. The method of claim 11 further comprising adding a third oligonucleotide including SEQ ID NO. 4 to the mixture.

14. The method of any of claim 4, 7, 10, or 13 wherein the third oligonucleotide comprises a label.

15. The method of claim 14 wherein the label comprises a fluorescent label.

16. The method of claim 15 wherein the fluorescent label is selected from the group consisting of FAM, dR110, 5-FAM, 6FAM, dR6G, JOE, HEX, VIC, TET, dTAMRA, TAMRA, NED, dROX, PET, BHQ+, Gold540, and LIZ.

17. The method of claim 1 wherein the first oligonucleotide is affixed to a solid substrate.

18. The method of claim 1 wherein the sample comprises an environmental sample.

19. The method of claim 1 wherein the sample is derived from a subject.

20. The method of claim 19 wherein the subject is a human.

21. The method of claim 19 wherein the subject is a companion animal.

22. The method of claim 19 wherein the subject is a livestock animal.

23. The method of claim 1 wherein the result comprises a nucleic acid sequence.

24. The method of claim 1 wherein the result comprises a ΔCt value.

25. A method of determining the presence or absence of C. posadasii in a sample, comprising:
adding an oligonucleotide including a sequence selected from the group consisting of SEQ ID NO. 5, SEQ ID NO. 6, SEQ ID NO. 7, SEQ ID NO. 8 SEQ ID NO. 9, SEQ ID NO. 10, SEQ ID NO. 11, SEQ ID NO. 12, SEQ ID NO. 13, SEQ ID NO. 14, SEQ ID NO. 15, and SEQ ID NO. 16 to a mixture comprising the sample;
subjecting the mixture to conditions that allow nucleic acid amplification; and
identifying the sample as containing C. posadasii or not containing C. posadasii based on a result of the nucleic acid amplification.

26. The method of claim 25 wherein the first oligonucleotide includes SEQ ID NO. 9, further comprising adding a second oligonucleotide including SEQ ID NO. 10 to the mixture.

27. The method of claim 26 further comprising performing DNA sequencing on a product of the nucleic acid amplification.

28. The method of claim 26 further comprising adding a third oligonucleotide including SEQ ID NO. 5 to the mixture.

29. The method of claim 25, wherein the first oligonucleotide includes SEQ ID NO. 11, further comprising adding a second nucleotide including SEQ ID NO. 12 to the mixture.

30. The method of claim 29 further comprising performing DNA sequencing on a product of the nucleic acid amplification.

31. The method of claim 29 further comprising adding a third oligonucleoide including SEQ ID NO. 6 to the mixture.

32. The method of claim 25 wherein the first oligonucleotide includes SEQ ID NO. 13, further comprising adding a second oligonucleotide including SEQ ID NO. 14 to the mixture.

33. The method of claim 32 further comprising performing DNA sequencing on a product of the nucleic acid amplification.

34. The method of claim 32 further comprising adding a third oligonucleotide including SEQ ID NO. 7 to the mixture.

35. The method of claim 25 wherein the first oligonucleotide includes SEQ ID NO. 15 further comprising adding a second oligonucleotide including SEQ ID NO. 16 to the mixture.

36. The method of claim 35 further comprising performing DNA sequencing on a product of the nucleic acid amplification.

37. The method of claim 35 further comprising adding a third oligonucleotide including SEQ ID NO. 8 to the mixture.

38. The method of any of claim 28, 31, 34, or 37 wherein the third oligonucleotide comprises a label.

39. The method of claim 38 wherein the label comprises a fluorescent label.

40. The method of claim 39 wherein the fluorescent label is selected from the group consisting of FAM, dR110, 5-FAM, 6FAM, dR6G, JOE, HEX, VIC, TET, dTAMRA, TAMRA, NED, dROX, PET, BHQ+, Gold540, and LIZ.

41. The method of claim 25 wherein the first oligonucleotide is affixed to a solid substrate.

42. The method of claim 25 wherein the sample comprises an environmental sample.

43. The method of claim 25 wherein the sample is derived from a subject.

44. The method of claim 43 wherein the subject is a human.

45. The method of claim 43 wherein the subject is a companion animal.

46. The method of claim 43 wherein the subject is a livestock animal.

47. The method of claim 25 wherein the result comprises sequence data.

48. The method of claim 25 wherein the result comprises a ΔCt value.

49. A method of determining whether a sample contains C. immitis or C. posadasii comprising:
receiving a sample;
isolating nucleic acid from the sample;
adding a first oligonucleotide including a sequence selected from the group consisting of SEQ ID NO. 9, SEQ ID NO. 11, SEQ ID NO. 13, and SEQ ID NO. 15 to a mixture comprising the nucleic acid;
adding a second oligonucleotide including a sequence selected from the group consisting of SEQ ID NO. 10, SEQ ID NO. 12, SEQ ID NO. 14, and SEQ ID NO. 16 to the mixture;
subjecting the mixture to conditions that allow for nucleic acid amplification identifying the sample as containing C. immitis or not containing C. immitis based upon a result of the nucleic acid amplification; and
identifying the sample as containing C. posadasii or not containing C. posadasii based upon a result of the nucleic acid amplification.

50. The method of claim 49 further comprising performing DNA sequencing upon a product of the nucleic acid amplification.

51. The method of claim 49 further comprising adding a third oligonucleotide including a sequence selected from the group consisting of SEQ ID NO. 1, SEQ ID NO. 2, SEQ ID NO. 3, and SEQ ID NO. 4 to the mixture and adding a fourth oligonucleotide including a sequence selected from the group consisting of SEQ ID NO. 5, SEQ ID NO. 6, SEQ ID NO. 7, and SEQ ID NO. 8 to the mixture; wherein the third oligonucleotide comprises a first fluorescent label and wherein the fourth oligonucleotide comprises a second fluorescent label and wherein the first fluorescent label and the second fluorescent label fluoresce at different wavelengths.

53. The method of claim 50 wherein the first oligonucleotide includes SEQ ID NO. 9 and the second oligonucleotide includes SEQ ID NO. 10.

54. The method of claim 51 wherein the first oligonucleotide includes SEQ ID NO. 9, the second oligonucleotide includes SEQ ID NO. 10, the third oligonucleotide includes SEQ ID NO. 1, and the fourth oligonucleotide includes SEQ ID NO. 5.

55. The method of claim 50 wherein the first oligonucleotide includes SEQ ID NO. 11 and the second oligonucleotide includes SEQ ID NO. 12.

56. The method of claim 51 wherein the first oligonucleotide includes SEQ ID NO. 11, the second oligonucleotide includes SEQ ID NO. 12, the third oligonucleotide includes SEQ ID NO. 2, and the fourth oligonucleotide includes SEQ ID NO. 6.

57. The method of claim 50 wherein the first oligonucleotide includes SEQ ID NO. 13 and the second oligonucleotide includes SEQ ID NO. 14.

58. The method of claim 51 wherein the first oligonucleotide includes SEQ ID NO. 13, the second oligonucleotide includes SEQ ID NO. 14, the third oligonucleotide includes SEQ ID NO. 3, and the fourth oligonucleotide includes SEQ ID NO. 7.

59. The method of claim 50 wherein the first oligonucleotide includes SEQ ID NO. 15 and the second oligonucleotide includes SEQ ID NO. 16.

60. The method of claim 51 wherein the first oligonucleotide includes SEQ ID NO. 15, the second oligonucleotide includes SEQ ID NO. 16, the third oligonucleotide includes SEQ ID NO. 4, and the fourth oligonucleotide includes SEQ ID NO. 8.

61. The method of claim 51 wherein the first fluorescent label and the second fluorescent label are each selected from the group consisting of FAM, dR110, 5-FAM, 6FAM, dR6G, JOE, HEX, VIC, TET, dTAMRA, TAMRA, NED, dROX, PET, BHQ+, Gold540, and LIZ.

62. The method of claim 49 wherein the first oligonucleotide is affixed to a solid substrate.

63. The method of claim 49 wherein the sample comprises an environmental sample.

64. The method of claim 49 wherein the sample is derived from a subject.

65. The method of claim 64 wherein the subject is a human.

66. The method of claim 64 wherein the subject is a companion animal.

67. The method of claim 64 wherein the subject is a livestock animal.

68. The method of claim 64 wherein the subject is suspected of having a Coccidioides infection.

69. The method of claim 49 wherein the result comprises a DNA sequence.

70. The method of claim 49 wherein the result comprises a ΔCt value.

71. A kit that facilitates determining whether a sample contains C. immitis or C. posadasii comprising:
a first oligonucleotide including a sequence selected from the group consisting of SEQ ID NO. 1, SEQ ID NO. 2, SEQ ID NO. 3, and SEQ ID NO. 4;
a second oligonucleotide including a sequence selected from the group consisting of SEQ ID NO. 5, SEQ ID NO. 6, SEQ ID NO. 7, and SEQ ID NO. 8;
and an indication of a result that signifies classification of the sample as containing a Coccidioides selected from the group consisting of C. immitis and C. posadasii.

72. The kit of claim 71 wherein the first oligonucleotide includes SEQ ID NO.1 and the second oligonucleotide includes SEQ ID NO. 5 further comprising a third oligonucleotide including a sequence selected from the group consisting of SEQ ID NO. 9 and SEQ ID NO. 10.

73. The kit of claim 71 wherein the first oligonucleotide includes SEQ ID NO. 2 and the second oligonucleotide includes SEQ ID NO. 6 further comprising a third oligonucleotide including a sequence selected from the group consisting of SEQ ID NO. 11 and SEQ ID NO. 12.

74. The kit of claim 71 wherein the first oligonucleotide includes SEQ ID NO. 3 and the second oligonucleotide includes SEQ ID NO. 7 further comprising a third oligonucleotide with a sequence selected from the group consisting of SEQ ID NO. 13 and SEQ ID NO. 14.

75. The kit of claim 71 wherein the first oligonucleotide includes SEQ ID NO. 4 and the second oligonucleotide includes SEQ ID NO. 8 further comprising a third oligonucleotide selected from the group consisting of SEQ ID NO. 15 and SEQ ID NO. 16.

76. The kit of claim 71 further comprising an enzyme.

77. The kit of claim 76 wherein the enzyme comprises a DNA polymerase.

78. The kit of claim 77 wherein the DNA polymerase is a thermostable DNA polymerase.

79. The kit of claim 71 wherein the first reagent is affixed to a substrate.

80. The kit of claim 71 further comprising a device to be used in collecting a sample.

81. The kit of claim 71 wherein the result comprises a ΔCt value.

82. The kit of claim 71 wherein the result comprises a nucleic acid sequence.

83. The kit of claim 71 wherein the indication comprises a positive control.

84. The kit of claim 71 wherein the indication comprises a writing.

85. The kit of claim 84 wherein the writing is physically included in the kit.

86. The kit of claim 84 wherein the writing is made available via a website.

87. The kit of claim 84 wherein the writing comprises an amplification plot.

88. The kit of claim 71 wherein the indication comprises software configured to detect the result as input and identification of the sample as containing C. immitis or C. posadasii as output.

89. The kit of claim 88 wherein the software is incorporated into a machine configured to detect fluorescence.


SEQUENCE LISTING (16 sequences)

SEQ ID NO: 1 - DNA, 18 bp, Coccidioides immitis
attgtccagtatgaggat

SEQ ID NO: 2 - DNA, 15 bp, Coccidioides immitis
aacatcaagacctcc

SEQ ID NO: 3 - DNA, 21 bp, Coccidioides immitis
aacgttgagataattacaggt

SEQ ID NO: 4 - DNA, 15 bp, Coccidioides immitis
acgccaatcagagca

SEQ ID NO: 5 - DNA, 18 bp, Coccidioides posadasii
attgtccagaatgaggat

SEQ ID NO: 6 - DNA, 13 bp, Coccidioides posadasii
catcaaggcctcc

SEQ ID NO: 7 - DNA, 20 bp, Cocciidioides posadasii
acgttgagataattacgggt

SEQ ID NO: 8 - DNA, 15 bp, Coccidioides posadasii
acgccaataagagca

SEQ ID NO: 9 - DNA, 21 bp, Coccidioides sp.
cgtgtggccttgcagtatagc

SEQ ID NO: 10 - DNA, 21 bp, Coccidioides sp.
tttacgccgtagcctttgatg

SEQ ID NO: 11 - DNA, 20 bp, Coccidioides sp.
aggccttctgcttggaatcc

SEQ ID NO: 12 - DNA, 24 bp, Coccidioides sp.
atattccgcaatttcaaggagaag

SEQ ID NO: 13 - DNA, 22 bp, Coccidioides sp.
ggatacgtcgctttccaactct

SEQ ID NO: 14 - DNA, 23 bp, Coccidioides sp.
cgctctatgtgatcgacacttga

SEQ ID NO: 15 - DNA, 20 bp, Coccidioides sp.
acgtgtccggctatttgcat

SEQ ID NO: 16 - DNA, 20 bp, Coccidioides sp.
cagctgtcgcggaataacct